(19) United States
(12) Patent Application Publication
(10) Pub. No.: US 2025/0194916 A1
(43) Pub. Date: Jun. 19, 2025

(54) DILATION INSTRUMENT WITH LATERALLY OFFSET GUIDE FEATURE

(71) Applicant: Acclarent, Inc., Irvine, CA (US)

(72) Inventors: Brittanie CHU, Chino Hill, CA (US); Sheila RUIZ, Missing Viejo, CA (US); Shubhayu BASU, Anaheim, CA (US); Henry F. SALAZAR, Pico Rivera, CA (US); Cesar FUENTES-ORTEGA, Pasadena, CA (US)

(21) Appl. No.: 18/986,664

(22) Filed: Dec. 18, 2024

Related U.S. Application Data
(60) Provisional application No. 63/611,232, filed on Dec. 18, 2023.

(51) Int. Cl. A61B 1/32; A61M 29/02

(52) U.S. Cl. CPC A61B 1/32; A61M 29/02

(57) ABSTRACT

A medical instrument can be used to dilate an anatomical passageway in a patient and position laterally offset guide features relevant to a dilation catheter to facilitate asymmetric dilation of passageways, targeted remodeling of anatomical structures, septoplasty, or other procedures. The medical instrument can have laterally offset features that include a body and a shaft assembly that extends distally relative to the body. The shaft assembly can include an outer sheath, a dilation catheter assembly, and a first guide feature that extends distally relative to the outer sheath. The dilation catheter assembly can include a dilator having a first longitudinal axis, and the first guide feature can have a second longitudinal axis that is laterally offset relative to the first longitudinal axis. The dilation catheter assembly can be translated along the first guide feature.

### CROSS-REFERENCE TO RELATED APPLICATIONS

This application claims the benefit of U.S. Provisional Application No. 63/611,232, filed Dec. 18, 2023, the entirety of which is incorporated herein by reference.

### BACKGROUND

There are various circumstances in which it may be necessary to dilate an anatomical passageway in a patient. For example, a patient may require the dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guidewire and catheter to position an inflatable balloon within the anatomical passageway, then inflating the balloon with a fluid (e.g., saline) to dilate the anatomical passageway.

For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pat. Nos. 11,534,192, 9,579,448, 9,155,492, and U.S. Pub. No. 2021/0361912, the entirety of each of which is incorporated by reference herein.

### SUMMARY OF THE INVENTIONS

In the context of Eustachian tube dilation, a dilation catheter or other dilation instrument may be inserted into the Eustachian tube and then inflated or otherwise expanded to dilate the Eustachian tube. The dilated Eustachian tube may provide improved ventilation from the nasopharynx to the middle ear and further provide improved drainage from the middle ear to the nasopharynx. Methods and devices for dilating the Eustachian tube are disclosed in U.S. Pat. Nos. 10,206,821 and 11,013,896, the entirety of each of which is incorporated herein by reference.

Some medical instruments may include an adjustable guide that allows the same medical instrument to readily access different anatomical structures (e.g., Eustachian tubes, different passageways associated with drainage of paranasal sinuses, etc.). Examples of dilation instruments with adjustable guides are described in U.S. Pat. Nos. 10,137,285, 11,013,897, and 11,534,192, the entirety of each of which is incorporated herein by reference.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images.

An example of an electromagnetic IGS navigation system that may be used in IGS procedures is the CARTO® 3 System by Biosense-Webster, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map.

During surgery, special instruments having sensors (e.g., electromagnetic coils that emit electromagnetic fields and/or are responsive to externally generated electromagnetic fields) are used to perform the procedure while the sensors send data to the computer indicating the current position of each surgical instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

In some scenarios, it may also be desirable to advance a dilation catheter along a guide that is laterally offset relative to the dilation catheter. While several systems and methods have been made and used to guide dilation catheters, it is believed that no one prior to the inventors has made or used the inventions described in the appended claims.

### BRIEF DESCRIPTION OF THE DRAWINGS

The drawings and detailed description that follow are intended to be merely illustrative and are not intended to limit the scope of the inventions as contemplated by the inventors.

FIG. 1A depicts a perspective view of an example of a medical instrument, with a dilation catheter in a proximal position, in accordance with some embodiments.

FIG. 1B depicts a perspective view of the medical instrument of FIG. 1A, with the dilation catheter in a distal position, and with a balloon of the dilation catheter in a non-inflated state, in accordance with some embodiments.

FIG. 1C depicts a perspective view of the medical instrument of FIG. 1A, with the dilation catheter in a distal position, and with the balloon of the dilation catheter in an inflated state, in accordance with some embodiments.

FIG. 2A depicts a side view of a distal portion of a shaft assembly of the medical instrument of FIG. 1A, with the dilation catheter in the proximal position, and with a guide element in a proximal position, in accordance with some embodiments.

FIG. 2B depicts a side view of a distal portion of the shaft assembly of FIG. 2A, with the dilation catheter in the proximal position, and with the guide element in a distal position, in accordance with some embodiments.

FIG. 2C depicts a side view of a distal portion of the shaft assembly of FIG. 2A, with the dilation catheter in the distal position, with the guide element in the distal position, and with the balloon of the dilation catheter in the non-inflated state, in accordance with some embodiments.

FIG. 2D depicts a side view of a distal portion of the shaft assembly of FIG. 2A, with the dilation catheter in the distal position, with the guide element in the distal position, and with the balloon of the dilation catheter in the inflated state, in accordance with some embodiments.

FIG. 3A depicts an end view of the shaft assembly of FIG. 2A, viewing from the distal end toward the proximal end, with the balloon of the dilation catheter in the non-inflated state, in accordance with some embodiments.

FIG. 3B depicts an end view of the shaft assembly of FIG. 2A, viewing from the distal end toward the proximal end, with the balloon of the dilation catheter in the inflated state, in accordance with some embodiments.

FIG. 4A depicts a side view of a distal portion of another shaft assembly that may be incorporated into the medical instrument of FIG. 1A, with a dilation catheter in a proximal position, and with a guide element in a proximal position, in accordance with some embodiments.

FIG. 4B depicts a side view of a distal portion of the shaft assembly of FIG. 4A, with the dilation catheter in the proximal position, and with the guide element in a distal position, in accordance with some embodiments.

FIG. 4C depicts a side view of a distal portion of the shaft assembly of FIG. 4A, with the dilation catheter in a distal position, with the guide element in the distal position, and with a balloon of the dilation catheter in the non-inflated state, in accordance with some embodiments.

FIG. 4D depicts a side view of a distal portion of the shaft assembly of FIG. 4A, with the dilation catheter in the distal position, with the guide element in the distal position, and with the balloon of the dilation catheter in the inflated state, in accordance with some embodiments.

FIG. 5A depicts an end view of the shaft assembly of FIG. 4A, viewing from the distal end toward the proximal end, with the balloon of the dilation catheter in the non-inflated state, in accordance with some embodiments.

FIG. 5B depicts an end view of the shaft assembly of FIG. 4A, viewing from the distal end toward the proximal end, with the balloon of the dilation catheter in the inflated state, in accordance with some embodiments.

FIG. 6A depicts a side view of a distal portion of another shaft assembly that may be incorporated into the medical instrument of FIG. 1A, with a dilation catheter in a proximal position, and with a guide element in a proximal position, in accordance with some embodiments.

FIG. 6B depicts a side view of a distal portion of the shaft assembly of FIG. 6A, with the dilation catheter in the proximal position, and with the guide element in a distal position, in accordance with some embodiments.

FIG. 6C depicts a side view of a distal portion of the shaft assembly of FIG. 6A, with the dilation catheter in a distal position, with the guide element in the distal position, and with a balloon of the dilation catheter in the non-inflated state, in accordance with some embodiments.

FIG. 6D depicts a side view of a distal portion of the shaft assembly of FIG. 6A, with the dilation catheter in the distal position, with the guide element in the distal position, and with the balloon of the dilation catheter in the inflated state, in accordance with some embodiments.

### DETAILED DESCRIPTION

The following description of certain examples of the inventions should not be used to limit the scope of the present inventions. Other examples, features, aspects, embodiments, and advantages of the inventions will become apparent to those skilled in the art from the following description, which is by way of illustration, one of the best modes contemplated for carrying out the inventions. As will be realized, the inventions are capable of other different and obvious aspects, all without departing from the inventions. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. EXAMPLE OF DILATION INSTRUMENT WITH SHAFT ASSEMBLY HAVING LATERALLY OFFSET FEATURES

In some scenarios, it may be desirable to advance a dilation catheter into an anatomical passageway in or near the ear, nose, or throat of a patient and expand the dilator to thereby dilate the passageway. For instance, it may be desirable to dilate a paranasal sinus ostium or other passageway associated with drainage of a paranasal sinus cavity, a Eustachian tube, a stenotic region in an airway of a patient, etc. It may also be desirable to incorporate one or more guide features into such an instrument, to assist in guiding the dilation catheter into the targeted anatomical passageway and to allow the dilation catheter to translate longitudinally relative to the guide feature. This may allow the guide feature to be initially positioned in relation to a targeted anatomical passageway while the dilation catheter is in a proximal position. The dilation catheter may then be advanced relative to the guide to a distal position to thereby enter the targeted anatomical passageway.

One or more guide features may be coaxially positioned with respect to a dilation catheter. However, in some scenarios, it may be beneficial to position the one or more guide features such that the one or more guide features are laterally offset with respect to the dilation catheter. For instance, having a guide feature be laterally offset with respect to a dilation catheter may facilitate asymmetric dilation of passageways (i.e., asymmetric about the central longitudinal axis of the dilation catheter), targeted remodeling of anatomical structures (e.g., turbinate crushing, posterior nasal nerve fracture, etc.), septoplasty, and/or other procedures. The following provides examples of ways in which a dilation instrument may incorporate one or more guide features that is/are laterally offset with respect to a dilation catheter.

A. Example of Dilation Instrument with Shaft Assembly having Laterally Offset Guide Feature and Coaxial Guide Feature

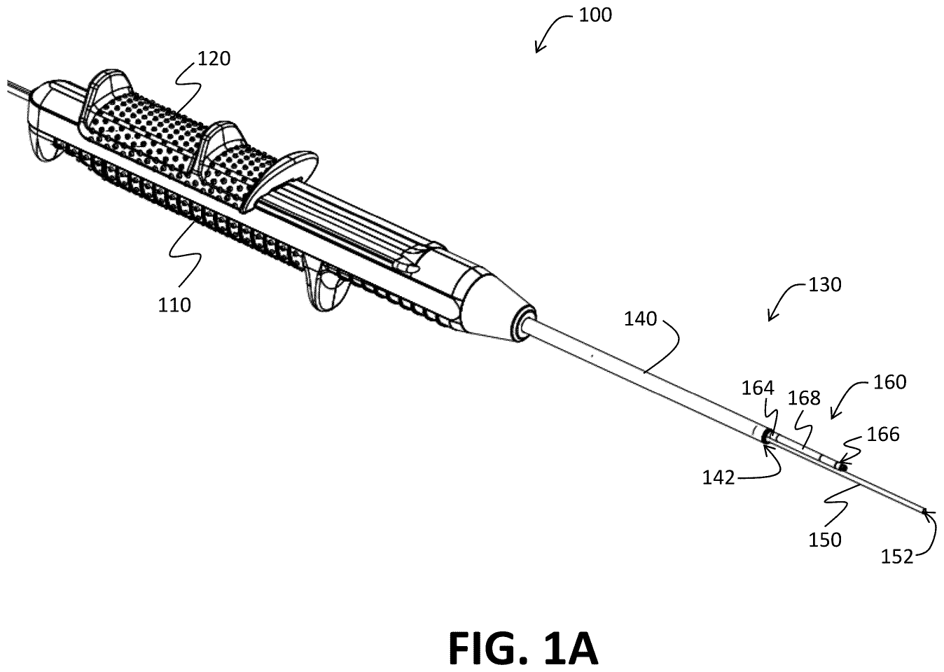

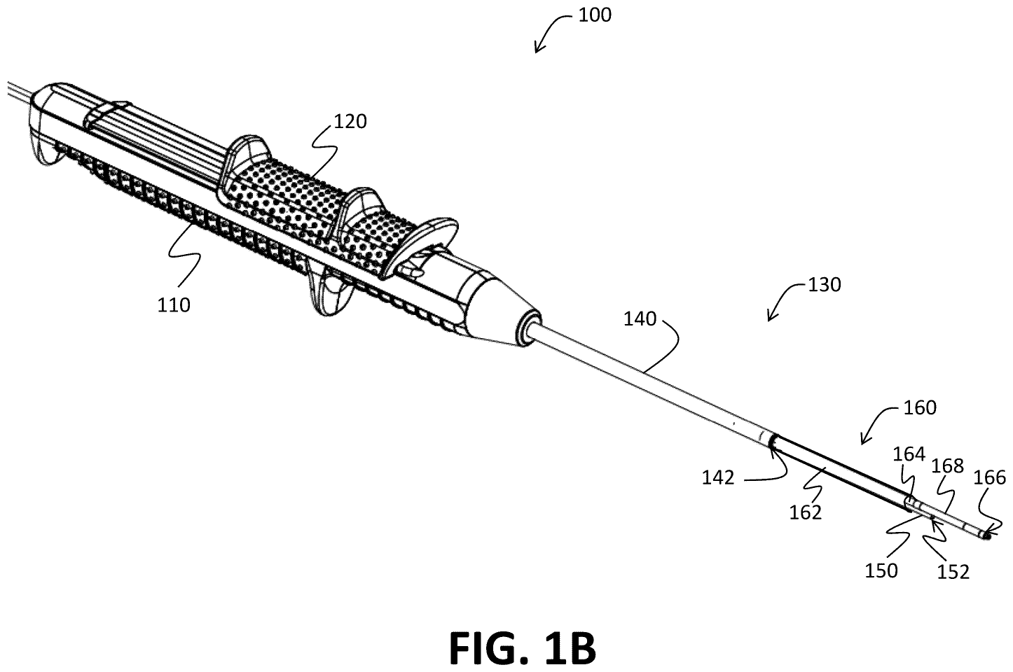

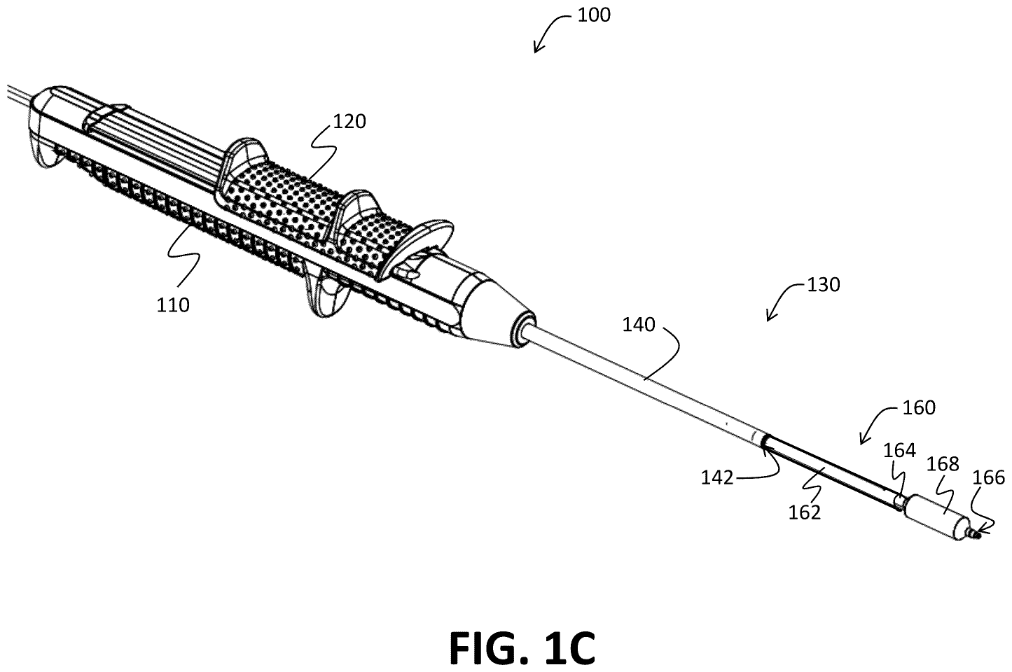

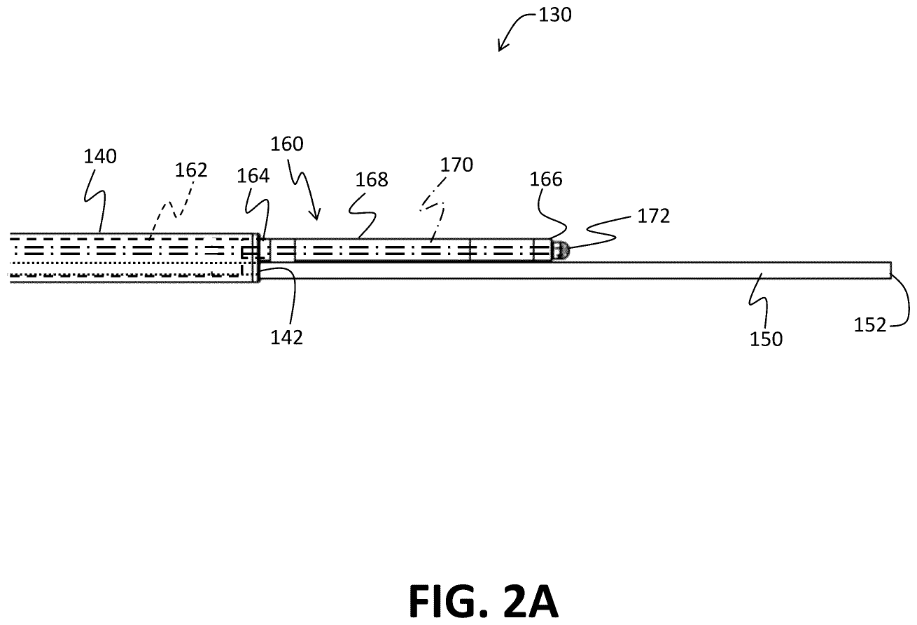

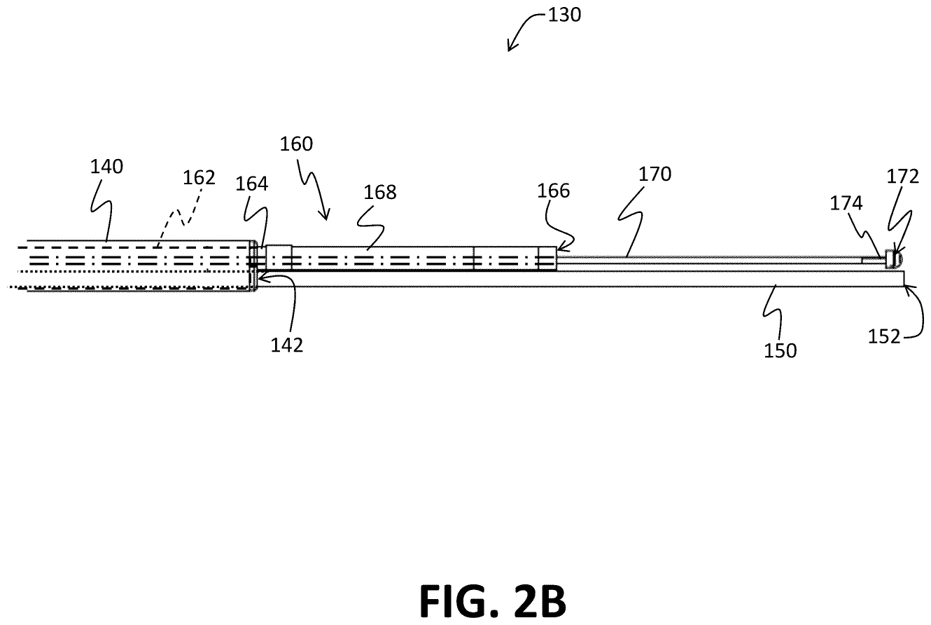

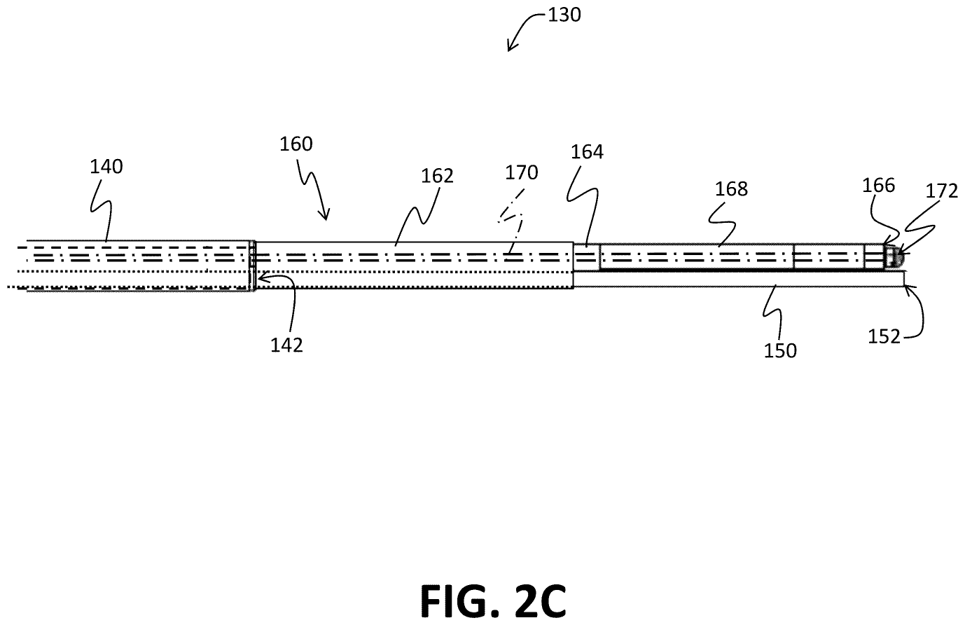

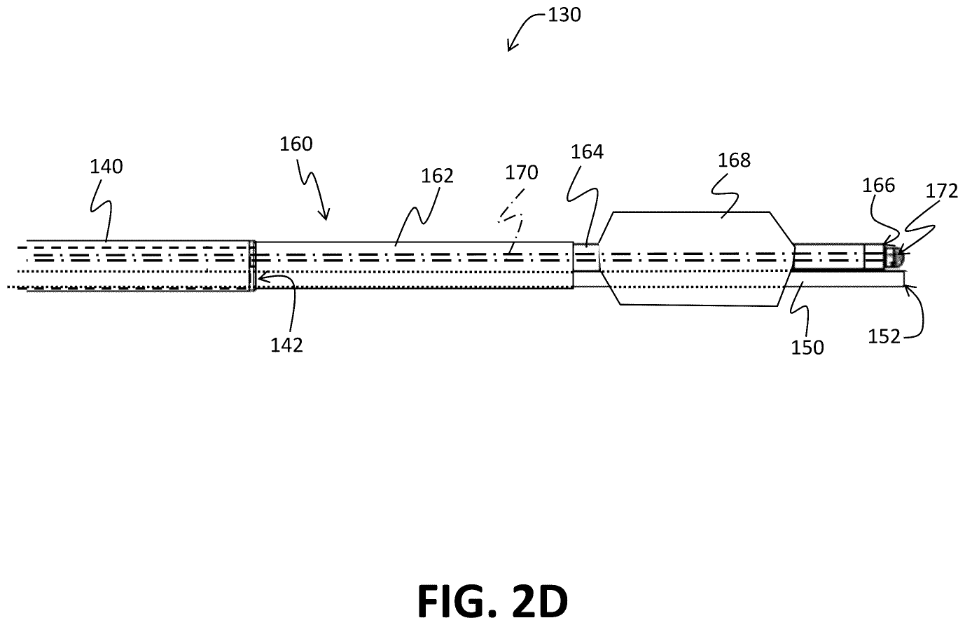

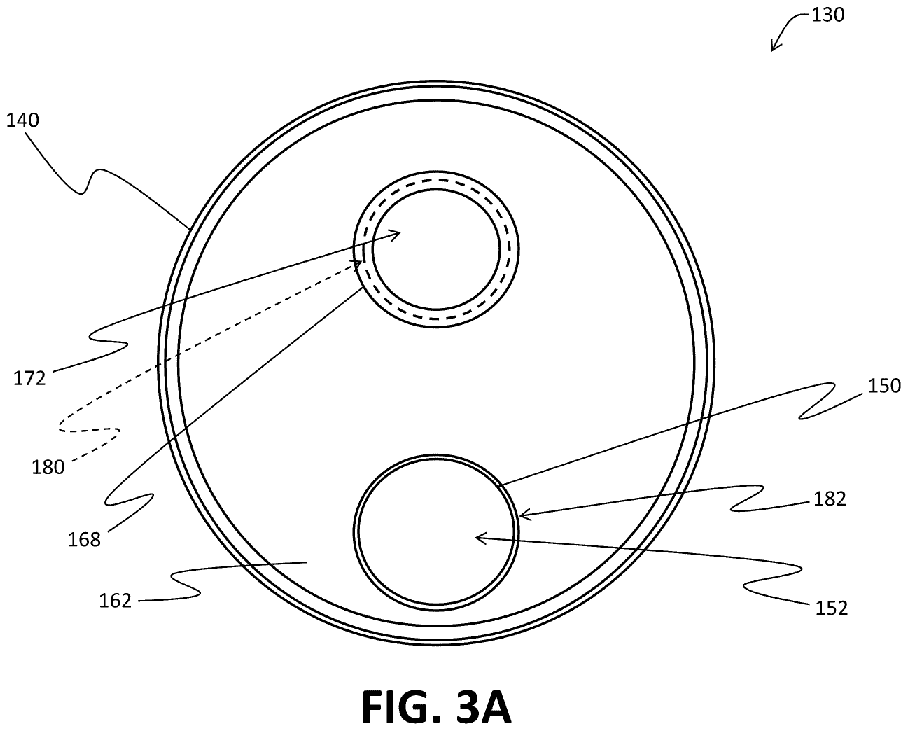

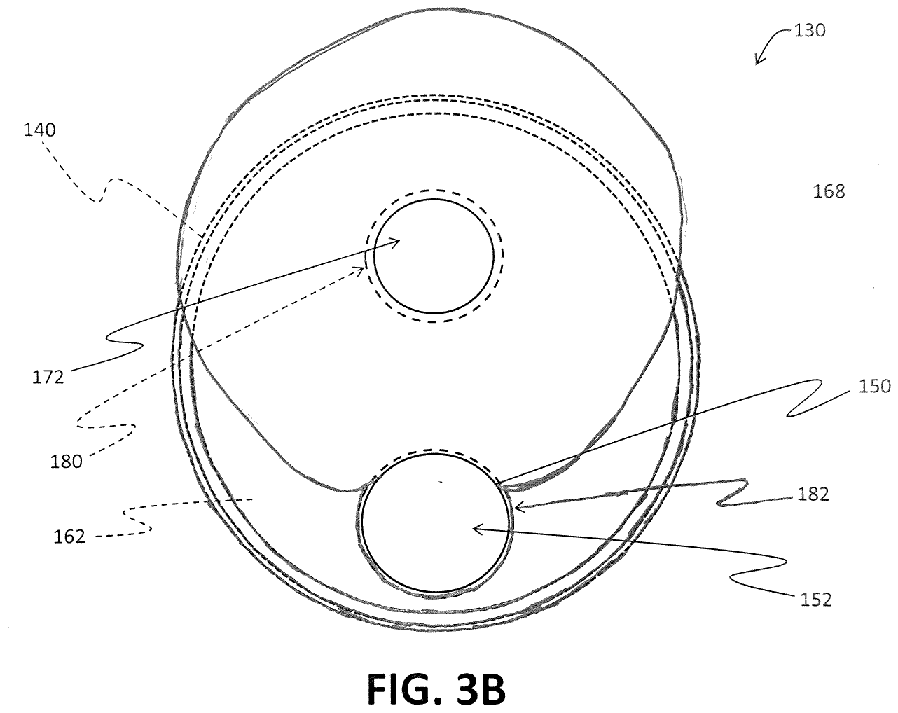

FIG. 1 shows an example of a dilation instrument 100 that is operable to perform various procedures in a head of a patient, including but not limited to dilation of an anatomical passageway (e.g., a paranasal sinus ostium, a frontal recess, a Eustachian tube, a meatus, etc.), remodeling of anatomical structures (e.g., turbinate crushing, posterior nasal nerve fracture, etc.), septoplasty, and/or other procedures. The dilation instrument 100 of this example includes a handle 110, an actuator 120, and a shaft assembly 130. As best seen in FIGS. 2A-3B, the shaft assembly 130 includes an outer sheath 140, a dilation catheter assembly 160, a first guide feature in the form of a guide rail 150, and a second guide feature in the form of a guide element 170. The outer sheath 140 comprises a rigid tube. Portions of the dilation catheter assembly 160 and the guide rail 150 extend distally from the outer sheath 140. In the present example, the outer sheath 140 does not enter the head of the patient during operation of the instrument 100, though some scenarios may exist where the outer sheath 140 enters the patient during operation of the instrument 100.

The actuator 120 is slidably supported on the handle 110, such that the actuator 120 is translatable between a proximal position (FIG. 1A) and a distal position (FIGS. 1B-1C) along the handle 110. The dilation catheter assembly 160 is coupled with the actuator 120, such that the dilation catheter assembly 160 also translates between a proximal position (FIG. 1A) and a distal position (FIGS. 1B-1C) with the actuator 120. The dilation catheter assembly 160 includes a proximal shaft portion 162 and a distal shaft portion 164 having a distal end 166. The proximal shaft portion 162 has an outer diameter that is larger than the outer diameter of the distal shaft portion 164. In the present example, the proximal shaft portion 162 is coaxial with the outer sheath 140. In some other versions, the proximal shaft portion 162 is laterally offset relative to a central longitudinal axis of the outer sheath 140.

As best seen in FIGS. 3A-3B, the proximal shaft portion 162 defines a first lumen 180 and a second lumen 182. The first lumen 180 further extends through the distal shaft portion 164. The guide element 170 is disposed in the first lumen 180 while the guide rail 150 is disposed in the second lumen 182. The first lumen 180 is coaxial with the distal shaft portion 164, which is laterally offset relative to a central longitudinal axis of the proximal shaft portion 162. In some other versions, the distal shaft portion 164 is coaxial with the central longitudinal axis of the proximal shaft portion 162. The second lumen 182 is laterally offset from the first lumen 180 and the distal shaft portion 164.

A balloon 168 is secured to the distal shaft portion 164 proximal to the distal end 166. The balloon 168 may comprise a non-extensible material and may be sized and configured to fit within a targeted anatomical passageway while in the deflated state (FIGS. 1A-1B, 2A-2C, and 3A) and dilate (or otherwise affect) the targeted anatomical passageway while in the inflated state (FIGS. 1C, 2D, and 3B).

The guide rail 150 of the present example comprises a malleable material and has an atraumatic distal tip 152. The guide rail 150 is fixedly secured to the outer sheath 140. In the present example, no portion of the guide rail 150 enters the handle 110 or is otherwise positioned proximally relative to a distal end of the handle 110 at any stage of operation of the instrument 100. The guide rail 150 is slidably disposed within the second lumen 182 of the proximal shaft portion 162 of the dilation catheter assembly 160, such that the dilation catheter assembly 160 is slidable along the guide rail 150 as described in greater detail below. The malleability of the guide rail 150 allows the guide rail 150 to be bent to a desired bend angle before being inserted into the head of the patient. The malleability of the guide rail 150 may allow the guide rail 150 to maintain the bend angle while the guide rail 150 is disposed in the head of the patient, including while the dilation catheter assembly 160 is advanced distally relative to the guide rail 150. Such operability of the guide rail 150 may promote access by the dilation catheter assembly 160 to various locations within the head of a patient, such as the maxillary sinus ostium, the frontal recess, the sphenoid sinus ostium, the Eustachian tube, etc., based on the selected bend angle.

The guide element 170 may be in the form of a flexible guidewire comprising metallic and/or polymeric material. In the present example, the guide element 170 has an enlarged distal tip 172 with an indicator element 174 that is configured to indicate the position of the distal tip 172 in three-dimensional space. In some versions, the guide element 170 is fixedly secured relative to the dilation catheter assembly 160, such that the distal tip 172 consistently remains positioned at the distal end 166 throughout operation of the dilation instrument 100. In some other versions, the guide element 170 is slidably disposed within the first lumen 180 of the dilation catheter assembly 160, such that the guide element 170 is translatable within the guide rail 150 (e.g., as shown in the transition from the state of operation shown in FIG. 2A to the state of operation shown in FIG. 2B). In some such versions, the dilation instrument 100 is configured to provide staged longitudinal translation of the guide element 170 and the dilation catheter assembly 160 in response to translation of the actuator 120 along the handle 110. In some other versions, a second actuator is provided to allow independent translation of the guide element 170. As yet another variation, the guide element 170 may instead be fixedly or slidably coaxially positioned within the guide rail 150. As yet another variation, the guide element 170 may be omitted.

In the present example, the indicator element 174 includes one or more position sensors that provide position-indicative signals to an IGS navigation system. Such position sensors may include one or more coils. When such a coil is positioned within an alternating electromagnetic field generated by field generators of the IGS navigation system, the alternating magnetic field may induce electrical current in the coil, and this induced electrical current may be communicated as a position-indicative signal via one or more wires, conductive traces, or other electrically conductive elements along the guide element 170. Signals from the indicator element 174 may be further communicated to a processor of the IGS navigation system, via wire or wirelessly. The processor may determine the location of the distal tip 172 within a three-dimensional space (e.g., within the head of the patient, etc.). To accomplish this, the processor may execute an algorithm to calculate location coordinates of the distal tip 172 from the position related signals (e.g., from induced currents) of the coil(s) of the indicator element 174. Thus, a position sensor of the indicator element 174 may generate signals indicating the real-time position of the sensor within three-dimensional space or by otherwise indicating the real-time position of the sensor within three-dimensional space.

In addition to including a position sensor, or as an alternative to including a position sensor, the indicator element 174 may include an illuminating feature that is operable to project light outwardly to thereby provide transillumination through the skin of the patient when the distal tip 172 is positioned inside a patient. In some such versions, the illuminating feature includes an optically transmissive window that is optically coupled with one or more optical fibers, with such one or more optical fibers being optically coupled with a light source. In some other versions, the illuminating feature includes one or more LEDs or other local sources of light positioned locally at the distal tip 172. It should be understood that the indicator element 174 may comprise a position sensor, an illuminating feature, or a combination of a position sensor and an illuminating feature.

FIGS. 1A and 2A show the shaft assembly 130 in a first state of operation, with the dilation catheter assembly 160 and the guide element 170 each in respective proximal positions. The distal end 162 and the distal tip 172 are thus both proximally positioned in relation to the distal tip 152 of the guide rail 150. In the present example, the proximal shaft portion 162 is positioned proximally in relation to the distal end 142 of the outer sheath 140 while the distal shaft portion 164 is positioned distally in relation to the distal end 142 of the outer sheath 140 at the initial stage of operation. In some versions, all or a portion of the distal shaft portion 164 is positioned proximally in relation to the distal end 142 of the outer sheath 140 in the initial stage of operation. In still other versions, a portion of the proximal shaft portion 162 is positioned distally in relation to the distal end 142 of the outer sheath 140 in the initial stage of operation.

With the shaft assembly 130 in the initial state of operation shown in FIGS. 1A and 2A, the distal portion of the shaft assembly 130 may be inserted in the head of the patient. While the guide rail 150 is shown in a straight configuration in the present example, the guide rail 150 may alternatively be in a bent configuration as noted above. The operator may select and execute the desired bend angle in the guide rail 150 based on the location and/or configuration of the targeted anatomical passageway. In versions where the guide element 170 is disposed within the guide rail 150, the real-time position feedback provided by the indicator element 174 may effectively indicate the real-time position of the distal tip 152 in three-dimensional space as the guide rail 150 and other components of the shaft assembly 130 are inserted into the patient. In some versions, as shown in FIG. 2B, the guide element 170 may be translated to a distal position as shown in FIG. 2B before, during, and/or after insertion of the shaft assembly 130 into the patient, whereby the real-time position feedback provided by the indicator element 174 may effectively indicate the real-time position of the distal tip 152 in three-dimensional space.

Once the operator has sufficiently positioned the shaft assembly 130 at the desired location, based on feedback from the indicator element 174 and/or based on other feedback, the operator may advance the actuator 120 distally along the handle 110 to the position shown in FIGS. 1B and 2C. The distal advancement of the actuator 120 drives the dilation catheter assembly 160 distally. When the dilation catheter assembly 160 is positioned distally, the proximal shaft portion 162 is positioned distally in relation to the distal end 142 of the outer sheath 140. With the guide rail 150 being slidably disposed in the second lumen 182 of the proximal shaft portion 162, the dilation catheter assembly 160 follows a path defined by the guide rail 150 as the dilation catheter assembly 160 translates from the proximal position to the distal position. Thus, if the guide rail 150 defines a bend, the dilation catheter assembly 160 will follow that bend as the dilation catheter assembly 160 translates from the proximal position to the distal position. While the guide rail 150 is malleable such that the guide rail 150 may be bent to achieve a desired bend on an ad hoc basis, the guide rail 150 has sufficient rigidity to maintain a formed bend as the dilation catheter assembly 160 translates along the bend.

In the present example, the distal tip 172 and the distal end 166 are positioned at approximately the same longitudinal position as the distal tip 152 when the dilation catheter assembly 160 is advanced to a distal position. In some versions, the dilation catheter assembly 160 may be advanced further distally, such that the distal end 166 is positioned distally in relation to the distal tip 152 when the dilation catheter assembly 160 is advanced to a distal position. In either arrangement, the balloon 168 may be positioned at the targeted anatomical region (e.g., a paranasal sinus ostium, a frontal recess, a Eustachian tube, a meatus, adjacent to a turbinate, adjacent to a deviated septum, etc.) when the dilation catheter assembly 160 is advanced to the distal position.

After the dilation catheter assembly 160 has been advanced to the distal position, the balloon 168 may be inflated as shown in FIGS. 1C and 2D. In the inflated state, the balloon 168 may affect the targeted anatomy. For instance, in the case of a paranasal sinus ostium, frontal recess, Eustachian tube, or meatus, the inflated the balloon 168 may dilate the anatomical passageway. In the case of a turbinate, the inflated the balloon 168 may crush a posterior nasal nerve in the turbinate and/or otherwise remodel the turbinate. In the case of a deviated septum, the inflated the balloon 168 may straighten the septum. In cases where the balloon 168 is coated with a drug or other material, the inflated the balloon 168 may apply that drug or other material to adjacent tissue. Alternatively, the inflated the balloon 168 may provide various other effects.

As shown in FIG. 3A, the balloon 168 is spaced away from the guide rail 150 when the balloon 168 is in a non-inflated state. As shown in FIG. 3B, the inflated the balloon 168 deforms around the guide rail 150, such that the balloon 168 inflates asymmetrically about a central longitudinal axis. In some scenarios, this asymmetric inflation may be beneficial. For instance, it may be desirable to have inflated the balloon 168 only bear against a certain anatomical structure while not simultaneously bearing against another anatomical structure, such that the asymmetric inflation may provide targeted anatomical structure remodeling, targeted drug delivery, and/or other targeted effects. In use, the operator may orient the shaft assembly 130 such that the guide rail 150 will be positioned at the anatomical region that the operator wishes to avoid bearing against with inflated the balloon 168. After the balloon 168 has been inflated and the desired effects achieved, the operator may deflate the balloon 168 and remove the shaft assembly 130 from the patient.

### B. First Example of Dilation Instrument Shaft Assembly Having Two Laterally Offset Guide Features

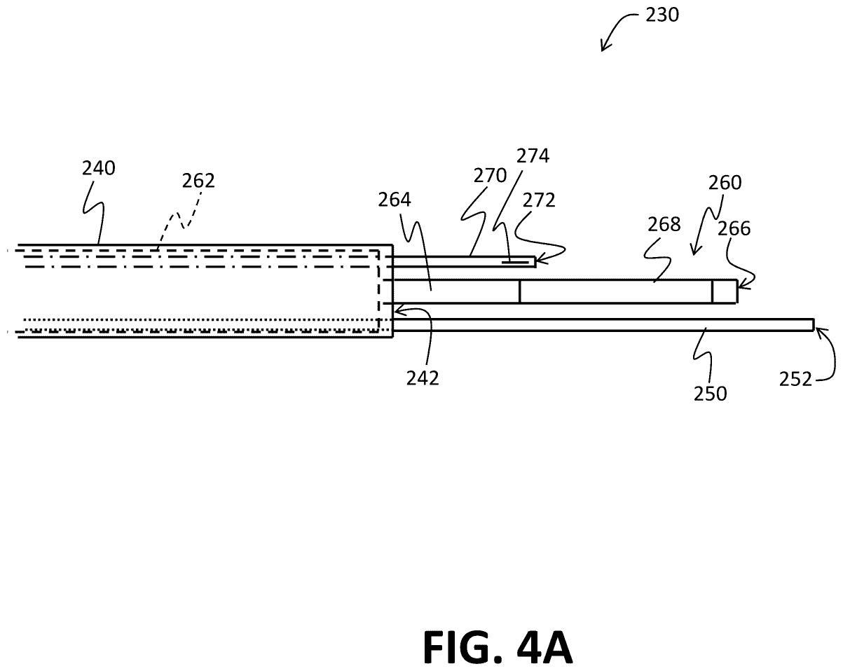

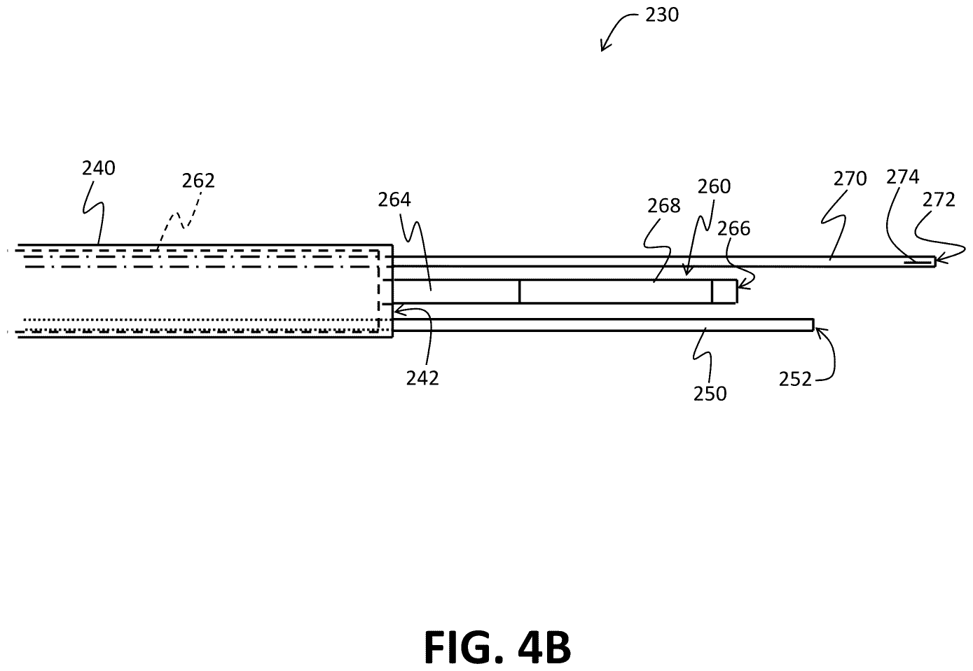

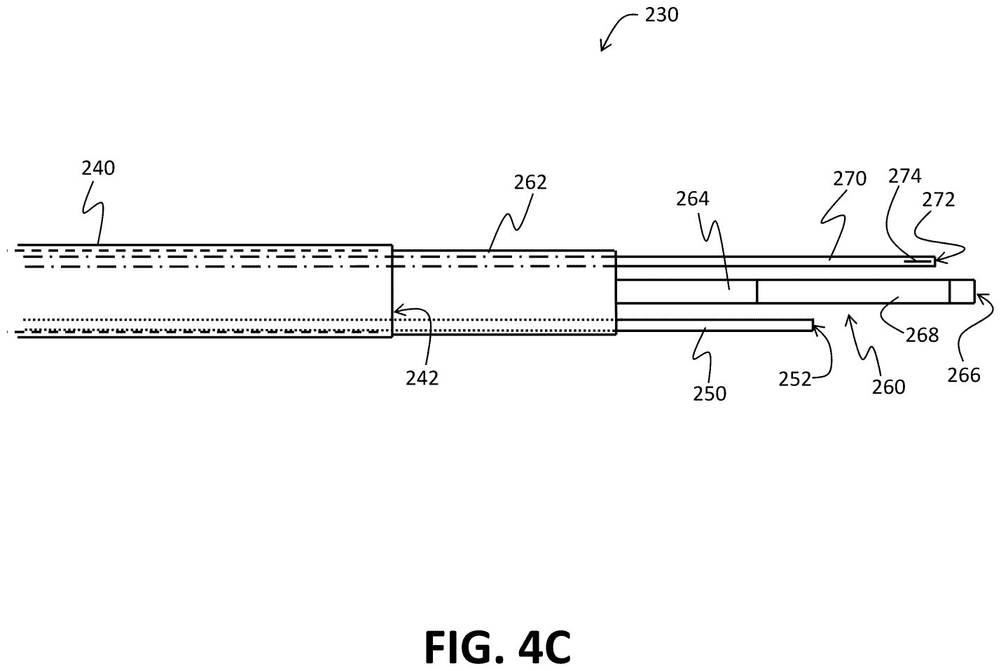

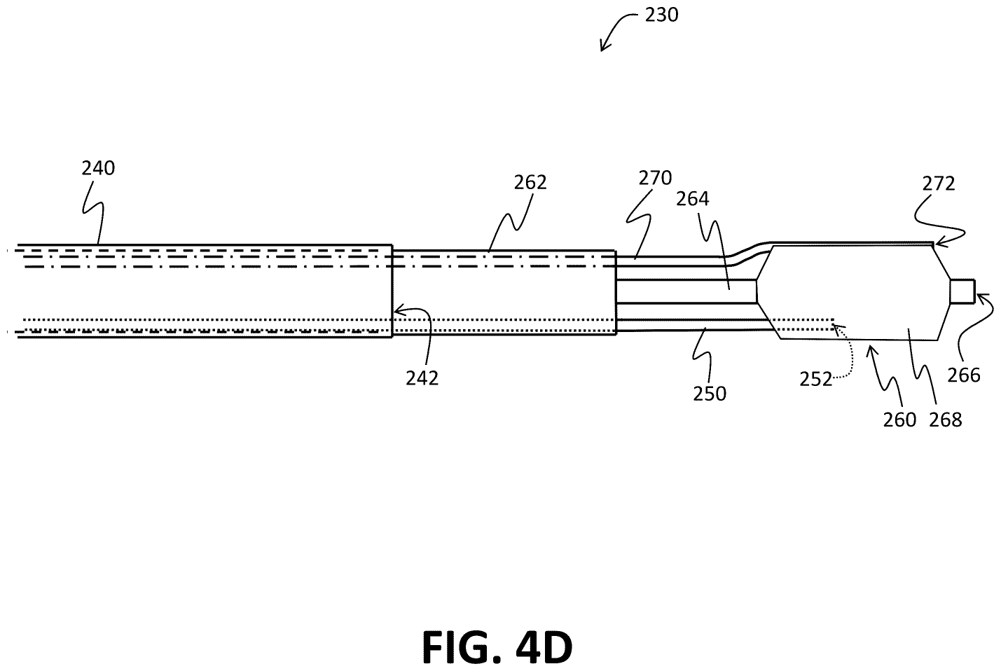

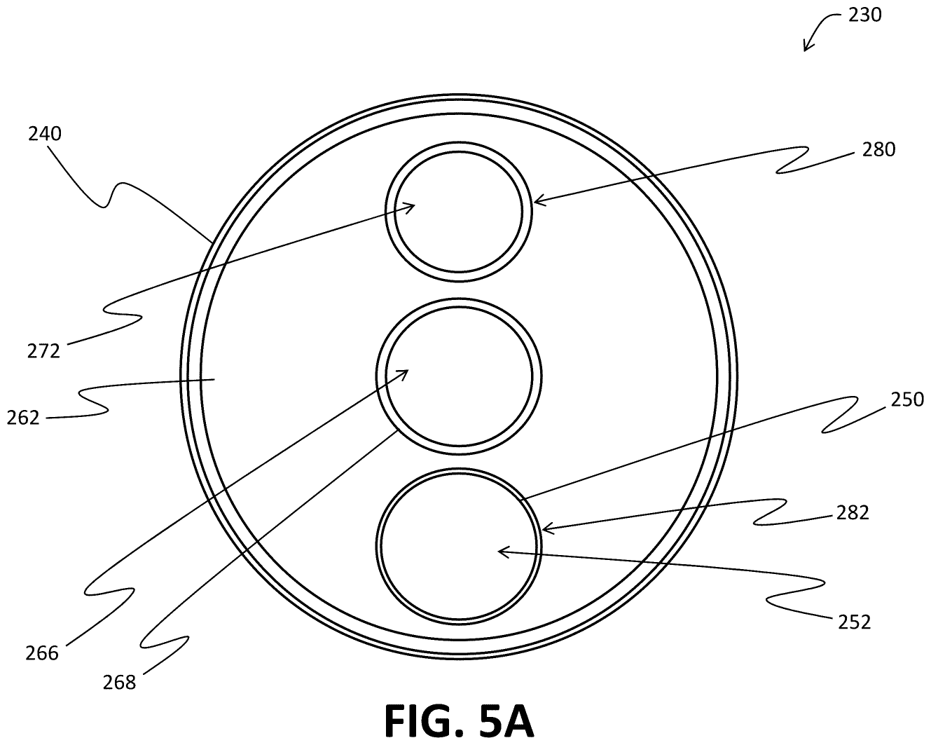

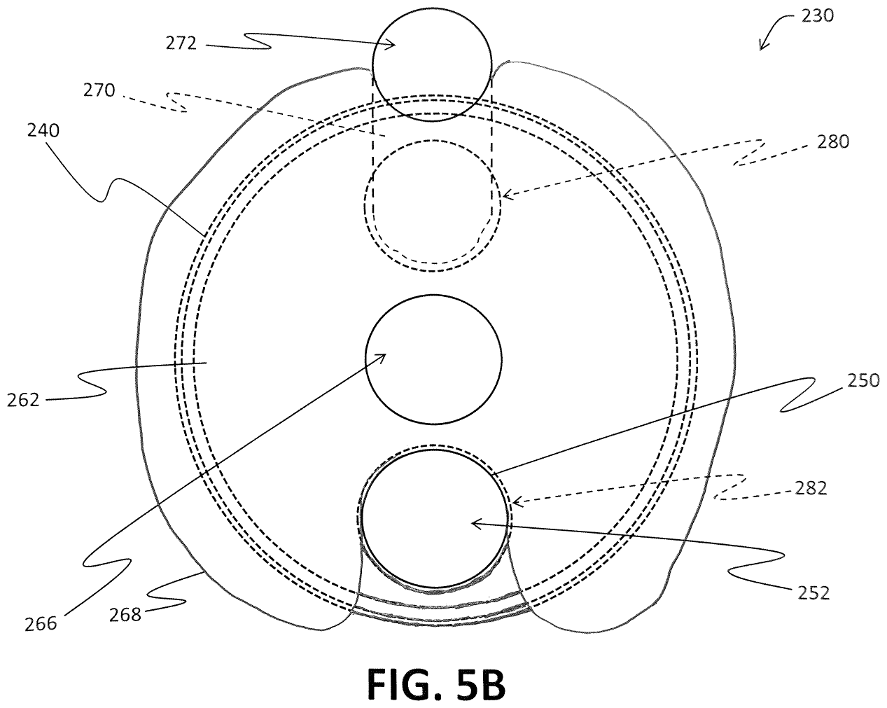

In accordance with at least some embodiments disclosed herein is the realization that it may be beneficial to position a first guide feature laterally offset from a dilation catheter assembly and also position a second guide feature laterally offset from a dilation catheter assembly. To that end, FIGS. 4A-5B show an example of another shaft assembly 230 that may be incorporated into the dilation instrument 100 in place of the shaft assembly 130. The shaft assembly 230 of this example may be configured and operable like the shaft assembly 130, except for the differences described below. The shaft assembly 230 of this example includes an outer sheath 240, a dilation catheter assembly 260, a first guide feature in the form of a guide rail 250, and a second guide feature in the form of a guide element 270. The outer sheath 240 comprises a rigid tube. Portions of the dilation catheter assembly 260, the guide rail 250, and the guide element 270 extend distally from the outer sheath 240. In the present example, the outer sheath 240 does not enter the head of the patient during operation of a variation of the instrument 100 incorporating the shaft assembly 230, though some scenarios may exist where the outer sheath 240 enters the patient during operation of a variation of the instrument 100 incorporating the shaft assembly 230.

The dilation catheter assembly 260 includes a proximal shaft portion 262 and a distal shaft portion 264 having a distal end 266. The proximal shaft portion 262 has an outer diameter that is larger than the outer diameter of the distal shaft portion 264. As best seen in FIGS. 5A-5B, the proximal shaft portion 262 defines a first lumen 280 and a second lumen 282. The guide element 270 is slidably disposed in the first lumen 280 while the guide rail 250 is slidably disposed in the second lumen 282. The first lumen 280 is laterally offset from the distal shaft portion 264, which is coaxial with the proximal shaft portion 262. The second lumen 282 is also laterally offset from first lumen 280 and the distal shaft portion 264. In the present example, the second lumen 282 is angularly spaced apart from first lumen 280 by about the central longitudinal axis of the proximal shaft portion 262 and the distal shaft portion 264 by 180 degrees. In other versions, the lumens 280, 282 may be angularly spaced apart from each other at any other suitable angle.

A balloon 268 is secured to the distal shaft portion 264 proximal to the distal end 266. The balloon 268 may comprise a non-extensible material and may be sized and configured to fit within a targeted anatomical passageway while in the deflated state (FIGS. 4A-4C and 5A) and dilate the targeted anatomical passageway while in the inflated state (FIGS. 4D and 5B).

The guide rail 250 of the present example comprises a malleable material and has an atraumatic distal tip 252. The guide rail 250 is fixedly secured to the outer sheath 240. In the present example, no portion of the guide rail 250 enters the handle 110 or is otherwise positioned proximally relative to a distal end of the handle 110 at any stage of operation of the instrument 100. The guide rail 250 is slidably disposed within the second lumen 282 of the proximal shaft portion 262 of the dilation catheter assembly 260, such that the dilation catheter assembly 260 is slidable along the guide rail 250 as described herein. The malleability of the guide rail 250 allows the guide rail 250 to be bent to a desired bend angle on an ad hoc basis before being inserted into the head of the patient. The malleability of the guide rail 250 may allow the guide rail 250 to maintain the bend angle while the guide rail 250 is disposed in the head of the patient, including while the dilation catheter assembly 260 is advanced distally relative to the guide rail 250. Such operability of the guide rail 250 may promote access by the dilation catheter assembly 260 to various locations within the head of a patient, such as the maxillary sinus ostium, the frontal recess, the sphenoid sinus ostium, the Eustachian tube, etc., based on the selected bend angle.

The guide element 270 may be in the form of a flexible guidewire comprising metallic and/or polymeric material. In the present example, the guide element 270 has a distal tip 272 with an indicator element 274 that is configured to indicate the position of the distal tip 272 in three-dimensional space. In some versions, the dilation instrument 100 is configured to provide staged longitudinal translation of the guide element 270 and the dilation catheter assembly 260 in response to translation of the actuator 120 along the handle 110. In some other versions, a second actuator is provided to allow independent translation of the guide element 270.

In the present example, the indicator element 274 includes one or more position sensors that provide position-indicative signals to an IGS navigation system. Such position sensors may include one or more coils. When such a coil is positioned within an alternating electromagnetic field generated by field generators of the IGS navigation system, the alternating magnetic field may induce electrical current in the coil, and this induced electrical current may be communicated as a position-indicative signal via one or more wires, conductive traces, or other electrically conductive elements along the guide element 270. Signals from the indicator element 274 may be further communicated to a processor of the IGS navigation system, via wire or wirelessly. The processor may determine the location of the distal tip 272 within a three-dimensional space (e.g., within the head of the patient, etc.). To accomplish this, the processor may execute an algorithm to calculate location coordinates of the distal tip 272 from the position related signals (e.g., from induced currents) of the coil(s) of the indicator element 274. Thus, a position sensor of the indicator element 274 may generate signals indicating the real-time position of the sensor within three-dimensional space or by otherwise indicating the real-time position of the sensor within three-dimensional space.

In addition to including a position sensor, or as an alternative to including a position sensor, the indicator element 274 may include an illuminating feature that is operable to project light outwardly to thereby provide transillumination through the skin of the patient when the distal tip 272 is positioned inside a patient. In some such versions, the illuminating feature includes an optically transmissive window that is optically coupled with one or more optical fibers, with such one or more optical fibers being optically coupled with a light source. In some other versions, the illuminating feature includes one or more LEDs or other local sources of light positioned locally at the distal tip 272. It should be understood that the indicator element 274 may comprise a position sensor, an illuminating feature, or a combination of a position sensor and an illuminating feature.

FIG. 4A shows the shaft assembly 230 in a first state of operation, with the dilation catheter assembly 260 and the guide element 270 each in respective proximal positions. The distal end 262 and the distal tip 272 are thus both proximally positioned in relation to the distal tip 252 of the guide rail 250. In the present example, the proximal shaft portion 262 is positioned proximally in relation to the distal end 242 of the outer sheath 240 while the distal shaft portion 264 is positioned distally in relation to the distal end 242 of the outer sheath 240 at the initial stage of operation. In some versions, all or a portion of the distal shaft portion 264 is positioned proximally in relation to the distal end 242 of the outer sheath 240 in the initial stage of operation. In still other versions, a portion of the proximal shaft portion 262 is positioned distally in relation to the distal end 242 of the outer sheath 240 in the initial stage of operation.

With the shaft assembly 230 in the initial state of operation shown in FIG. 4A, the distal portion of the shaft assembly 230 may be inserted in the head of the patient. While the guide rail 250 is shown in a straight configuration in the present example, the guide rail 250 may alternatively be in a bent configuration as noted above. The operator may select and execute the desired bend angle in the guide rail 250 based on the location and/or configuration of the targeted anatomical passageway. In versions where a position sensor is integrated into the distal tip 252 of the guide rail 250, the real-time position feedback provided by that position sensor may effectively indicate the real-time position of the distal tip 252 in three-dimensional space as the guide rail 250 and other components of the shaft assembly 230 are inserted into the patient. In addition, or in the alternative, the guide element 270 may be translated to a distal position as shown in FIG. 4B before, during, and/or after insertion of the shaft assembly 230 into the patient, whereby the real-time position feedback provided by the indicator element 274 may effectively indicate the real-time position of the distal tip 252 and/or another portion of the shaft assembly 230 in three-dimensional space.

Once the operator has sufficiently positioned the shaft assembly 230 at the desired location, based on feedback from the indicator element 274 and/or based on other feedback, the operator may advance the dilation catheter assembly 260 distally. As shown in FIG. 4C, when the dilation catheter assembly 260 is positioned distally, the proximal shaft portion 262 is positioned distally in relation to the distal end 242 of the outer sheath 240. With the guide rail 250 being slidably disposed in the second lumen 282 of the proximal shaft portion 262, the dilation catheter assembly 260 follows a path defined by the guide rail 250 as the dilation catheter assembly 260 translates from the proximal position to the distal position. Thus, if the guide rail 250 defines a bend, the dilation catheter assembly 260 will follow that bend as the dilation catheter assembly 260 translates from the proximal position to the distal position. While the guide rail 250 is malleable such that the guide rail 250 may be bent to achieve a desired bend, the guide rail 250 has sufficient rigidity to maintain a formed bend as the dilation catheter assembly 260 translates along the bend.

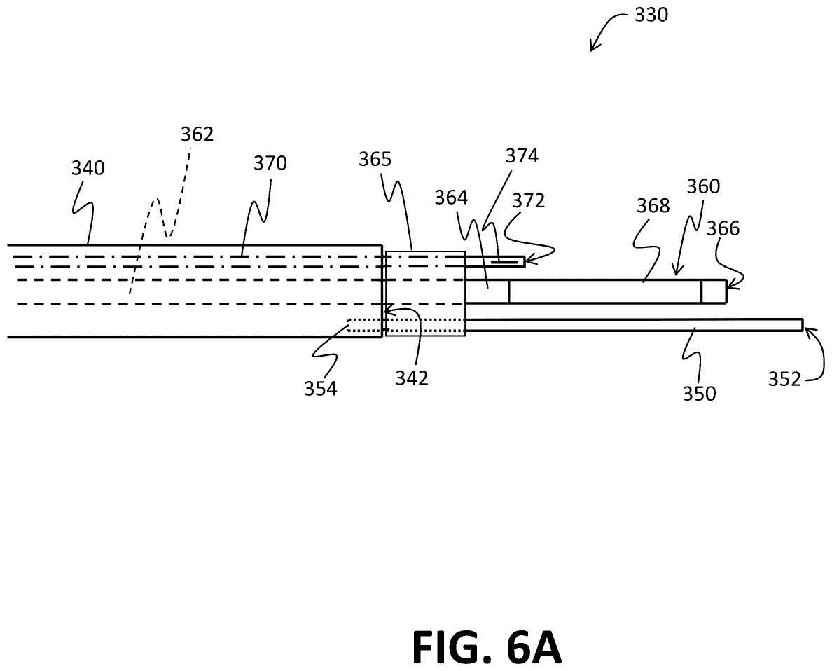

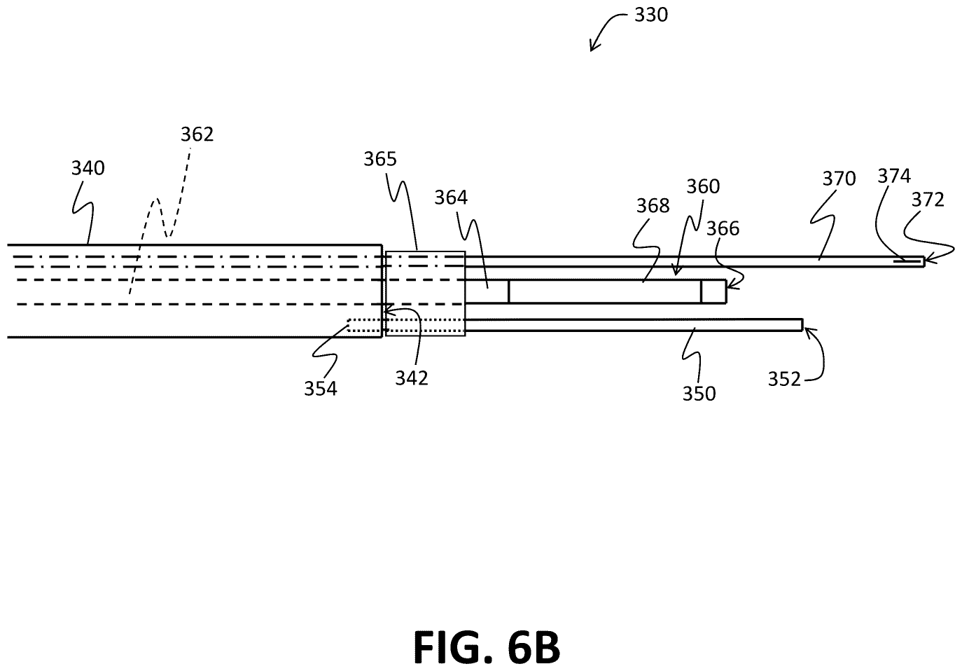

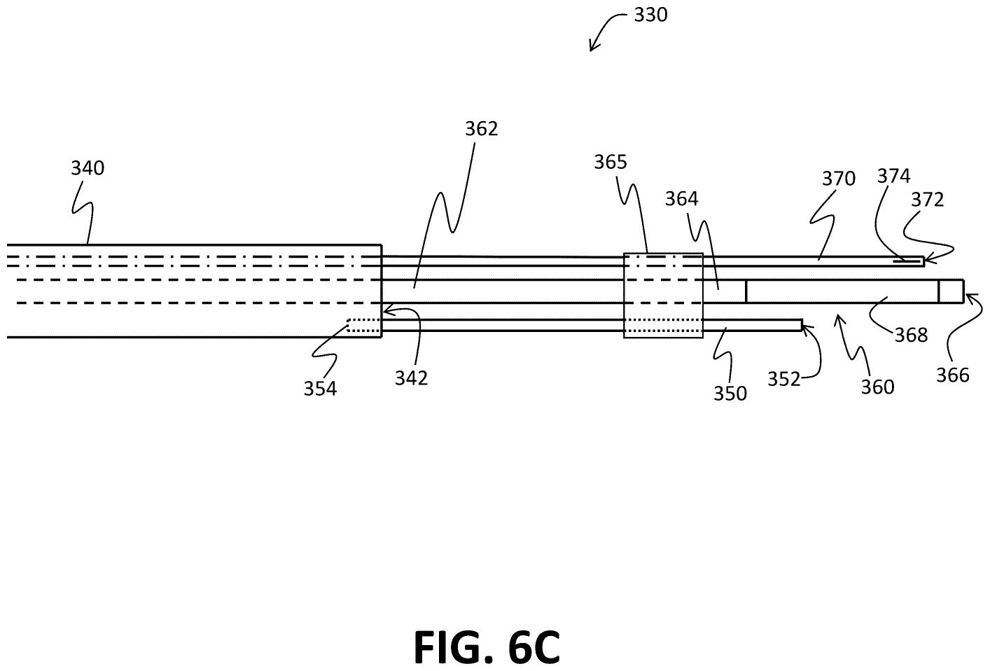

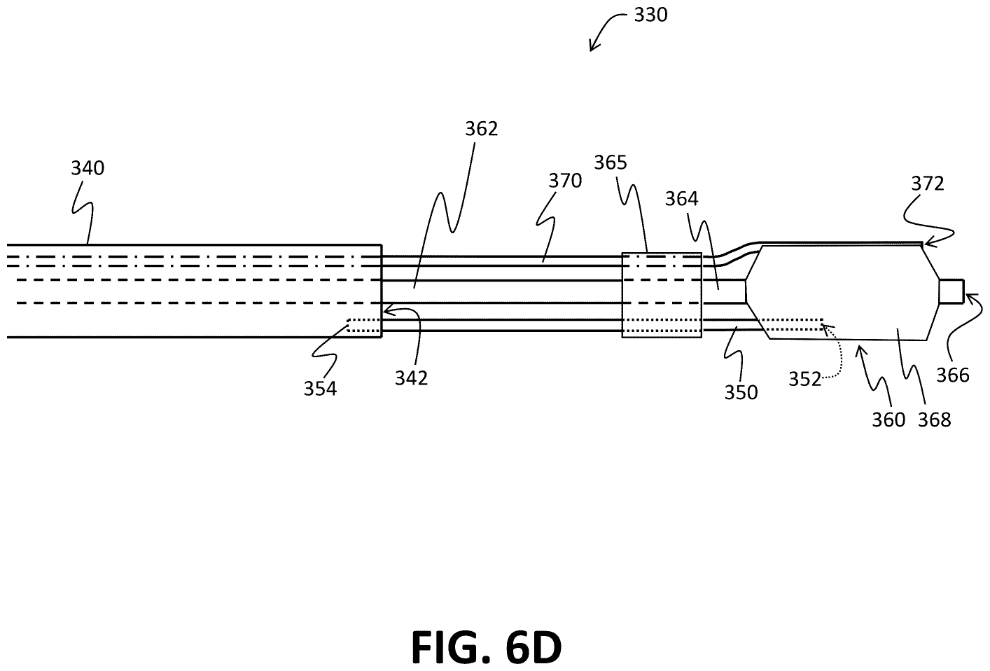

In the present example, the distal tip 272 may be positioned to approximate the position of the distal end 266 when the dilation catheter assembly 260 is advanced to a distal position. In some versions, the dilation catheter assembly 260 may be advanced to a point where the distal end 266 is also located at approximately the same longitudinal position as the distal tip 252. Alternatively, as shown in FIG. 6C, the dilation catheter assembly 260 may be advanced to a point where the distal end 266 is positioned distally in relation to the distal tip 252 when the dilation catheter assembly 260 is advanced to a distal position, as shown in FIG. 4C. In any of these scenarios, the balloon 268 may be positioned at the targeted anatomical region (e.g., a paranasal sinus ostium, a frontal recess, a Eustachian tube, a meatus, adjacent to a turbinate, adjacent to a deviated septum, etc.) when the dilation catheter assembly 260 is advanced to the distal position.

After the dilation catheter assembly 260 has been advanced to the distal position, the balloon 268 may be inflated as shown in FIG. 4D. In the inflated state, the balloon 268 may affect the targeted anatomy. For instance, in the case of a paranasal sinus ostium, frontal recess, Eustachian tube, or meatus, the inflated the balloon 268 may dilate the anatomical passageway. In the case of a turbinate, the inflated the balloon 268 may crush a posterior nasal nerve in the turbinate and/or otherwise remodel the turbinate. In the case of a deviated septum, the inflated the balloon 268 may straighten the septum. In cases where the balloon 268 is coated with a drug or other material, the inflated the balloon 268 may apply that drug or other material to adjacent tissue. Alternatively, the inflated the balloon 268 may provide various other effects.

As shown in FIG. 5A, the balloon 268 is spaced away from the guide rail 250 when the balloon 268 is in a non-inflated state. As shown in FIG. 5B, the inflated the balloon 268 deforms around the guide rail 250, such that the balloon 268 inflates asymmetrically about a central longitudinal axis. In accordance with at least some embodiments disclosed herein is the realization that this asymmetric inflation may be beneficial, as described above with respect to the asymmetric inflation of the balloon 168. As also shown in FIGS. 4D and 5B, flexibility in the guide element 270 may allow the guide element 270 to deform when the balloon 268 is inflated and thus bears against the guide element 270. As also best seen in FIG. 5B, the balloon 268 may also deform to some degree around the guide element 270 when the balloon 268 is inflated. In some other versions, the guide element 270 does not deform when the balloon 268 is inflated, such that the guide element 270 maintains a substantially straight configuration when the balloon 268 is inflated. In still other versions, the balloon 268 does not deform around the guide element 270 when the balloon 268 is inflated. In any of these scenarios, after the balloon 268 has been inflated and the desired effects achieved, the operator may deflate the balloon 268 and remove the shaft assembly 230 from the patient.

### C. Second Example of Dilation Instrument Shaft Assembly Having Two Laterally Offset Guide Features

FIGS. 6A-6D show another example of another shaft assembly 330 that may be incorporated into the dilation instrument 100 in place of the shaft assembly 130. The shaft assembly 330 of this example may be configured and operable like the shaft assembly 130 and/or the shaft assembly 230, except for the differences described below. The shaft assembly 330 of this example includes an outer sheath 340, a dilation catheter assembly 360, a first guide feature in the form of a guide rail 350, and a second guide feature in the form of a guide element 370. The outer sheath 340 comprises a rigid tube. Portions of the dilation catheter assembly 360, the guide rail 350, and the guide element 370 extend distally from the outer sheath 340. In the present example, the outer sheath 340 does not enter the head of the patient during operation of a variation of the instrument 100 incorporating the shaft assembly 330, though some scenarios may exist where the outer sheath 340 enters the patient during operation of a variation of the instrument 100 incorporating the shaft assembly 330.

The dilation catheter assembly 360 includes a proximal shaft portion 362, a distal shaft portion 364 having a distal end 366, and a hub 365 interposed between the shaft portions 362, 364. In the present example, the shaft portions 362, 364 are together defined by a single, continuous tubular structure that passes through a central region of the hub 365, such that the shaft portions 362, 364 and the hub 365 are coaxially aligned with each other. In such versions, the hub 365 may be fixedly secured to the single, continuous tubular structure that defines the shaft portions 362, 364. Also in the present example, the proximal shaft portion 362 has the same outer diameter as the distal shaft portion 364, while the hub 365 has a larger outer diameter. In other versions, any of these structural relationships may be varied. For instance, in some other versions the shaft portions 362, 364 are formed by two separate tubular structures that are each coupled with the hub 365. In some such versions, these two separate tubular structures are not coaxially aligned with each other, such that the proximal shaft portion 362 may be laterally offset relative to the distal shaft portion 364. Alternatively, any other suitable structural configurations and arrangements may be provided.

The hub 365 defines a first lumen that is analogous to the first lumen 280 and a second lumen that is analogous to the second lumen 282. The guide element 370 is slidably disposed in the first lumen while the guide rail 350 is slidably disposed in the second lumen. The first and second lumens of the hub 365 may otherwise be configured, operable, and varied in accordance with the above description of the first and second lumens 280, 282.

A balloon 368 is secured to the distal shaft portion 364 proximal to the distal end 366. The balloon 368 may comprise a non-extensible material and may be sized and configured to fit within a targeted anatomical passageway while in the deflated state (FIGS. 6A-6C) and dilate the targeted anatomical passageway while in the inflated state (FIG. 6D).

The guide rail 350 of the present example comprises a malleable material and has an atraumatic distal tip 352. In the present example, the guide rail 350 is configured and positioned such that a proximal end 354 of the guide rail 350 is positioned near the distal end 342 of the outer sheath 340 and such that the guide rail 350 is fixedly secured to the outer sheath 340 (e.g., via welding, epoxy, adhesive, etc.). In the present example, no portion of the guide rail 350 enters the handle 110 or is otherwise positioned proximally relative to a distal end of the handle 110 at any stage of operation of the instrument 100. The guide rail 350 is slidably disposed within the second lumen 382 of the hub 365 of the dilation catheter assembly 360, such that the dilation catheter assembly 360 is slidable along the guide rail 350 as described herein. The malleability of the guide rail 350 allows the guide rail 350 to be bent to a desired bend angle on an ad hoc basis before being inserted into the head of the patient. The malleability of the guide rail 350 may allow the guide rail 350 to maintain the bend angle while the guide rail 350 is disposed in the head of the patient, including while the dilation catheter assembly 360 is advanced distally relative to the guide rail 350. Such operability of the guide rail 350 may promote access by the dilation catheter assembly 360 to various locations within the head of a patient, such as the maxillary sinus ostium, the frontal recess, the sphenoid sinus ostium, the Eustachian tube, etc., based on the selected bend angle.

The guide element 370 may be in the form of a flexible guidewire comprising metallic and/or polymeric material. In the present example, the guide element 370 has a distal tip 372 with an indicator element 374 that is configured to indicate the position of the distal tip 372 in three-dimensional space. In some versions, the dilation instrument 100 is configured to provide staged longitudinal translation of the guide element 370 and the dilation catheter assembly 360 in response to translation of the actuator 120 along the handle 110. In some other versions, a second actuator is provided to allow independent translation of the guide element 370.

In the present example, the indicator element 374 includes one or more position sensors that provide position-indicative signals to an IGS navigation system. Such position sensors may include one or more coils. When such a coil is positioned within an alternating electromagnetic field generated by field generators of the IGS navigation system, the alternating magnetic field may induce electrical current in the coil, and this induced electrical current may be communicated as a position-indicative signal via one or more wires, conductive traces, or other electrically conductive elements along the guide element 370. Signals from the indicator element 374 may be further communicated to a processor of the IGS navigation system, via wire or wirelessly. The processor may determine the location of the distal tip 372 within a three-dimensional space (e.g., within the head of the patient, etc.). To accomplish this, the processor may execute an algorithm to calculate location coordinates of the distal tip 372 from the position related signals (e.g., from induced currents) of the coil(s) of the indicator element 374. Thus, a position sensor of the indicator element 374 may generate signals indicating the real-time position of the sensor within three-dimensional space or by otherwise indicating the real-time position of the sensor within three-dimensional space.

In addition to including a position sensor, or as an alternative to including a position sensor, the indicator element 374 may include an illuminating feature that is operable to project light outwardly to thereby provide transillumination through the skin of the patient when the distal tip 372 is positioned inside a patient. In some such versions, the illuminating feature includes an optically transmissive window that is optically coupled with one or more optical fibers, with such one or more optical fibers being optically coupled with a light source. In some other versions, the illuminating feature includes one or more LEDs or other local sources of light positioned locally at the distal tip 372. It should be understood that the indicator element 374 may comprise a position sensor, an illuminating feature, or a combination of a position sensor and an illuminating feature.

FIG. 6A shows the shaft assembly 330 in a first state of operation, with the dilation catheter assembly 360 and the guide element 370 each in respective proximal positions. The distal end 366 and the distal tip 372 are, thus, both proximally positioned in relation to the distal tip 352 of the guide rail 350. In the present example, the proximal shaft portion 362 is positioned proximally in relation to the distal end 342 of the outer sheath 340 while the distal shaft portion 364 and the hub 365 are positioned distally in relation to the distal end 342 of the outer sheath 340 at the initial stage of operation. In some versions, all or a portion of the distal shaft portion 364 and/or the hub 365 is/are positioned proximally in relation to the distal end 342 of the outer sheath 340 in the initial stage of operation. In still other versions, a portion of the proximal shaft portion 362 is positioned distally in relation to the distal end 342 of the outer sheath 340 in the initial stage of operation.

With the shaft assembly 330 in the initial state of operation shown in FIG. 6A, the distal portion of the shaft assembly 330 may be inserted in the head of the patient. While the guide rail 350 is shown in a straight configuration in the present example, the guide rail 350 may alternatively be in a bent configuration as noted above. The operator may select and execute the desired bend angle in the guide rail 350 based on the location and/or configuration of the targeted anatomical passageway. In versions where a position sensor is integrated into the distal tip 352 of the guide rail 350, the real-time position feedback provided by that position sensor may effectively indicate the real-time position of the distal tip 352 in three-dimensional space as the guide rail 350 and other components of the shaft assembly 330 are inserted into the patient. In addition, or in the alternative, the guide element 370 may be translated to a distal position as shown in FIG. 6B before, during, and/or after insertion of the shaft assembly 330 into the patient, whereby the real-time position feedback provided by the indicator element 374 may effectively indicate the real-time position of the distal tip 352 and/or another portion of the shaft assembly 330 in three-dimensional space.

Once the operator has sufficiently positioned the shaft assembly 330 at the desired location, based on feedback from the indicator element 374 and/or based on other feedback, the operator may advance the dilation catheter assembly 360 distally. As shown in FIG. 6C, when the dilation catheter assembly 360 is positioned distally, the proximal shaft portion 362 is positioned distally in relation to the distal end 342 of the outer sheath 340. With the guide rail 350 being slidably disposed in the second lumen 382 of the proximal shaft portion 362, the dilation catheter assembly 360 follows a path defined by the guide rail 350 as the dilation catheter assembly 360 translates from the proximal position to the distal position. Thus, if the guide rail 350 defines a bend, the dilation catheter assembly 360 will follow that bend as the dilation catheter assembly 360 translates from the proximal position to the distal position. While the guide rail 350 is malleable such that the guide rail 350 may be bent to achieve a desired bend, the guide rail 350 has sufficient rigidity to maintain a formed bend as the dilation catheter assembly 360 translates along the bend.

In the present example, the distal tip 372 may be positioned to approximate the position of the distal end 366 when the dilation catheter assembly 360 is advanced to a distal position. In some versions, the dilation catheter assembly 360 may be advanced to a point where the distal end 366 is also located at approximately the same longitudinal position as the distal tip 352. Alternatively, as shown in FIG. 4C, the dilation catheter assembly 360 may be advanced to a point where the distal end 366 is positioned distally in relation to the distal tip 352 when the dilation catheter assembly 360 is advanced to a distal position, as shown in FIG. 4C. In any of these scenarios, the balloon 368 may be positioned at the targeted anatomical region (e.g., a paranasal sinus ostium, a frontal recess, a Eustachian tube, a meatus, adjacent to a turbinate, adjacent to a deviated septum, etc.) when the dilation catheter assembly 360 is advanced to the distal position.

After the dilation catheter assembly 360 has been advanced to the distal position, the balloon 368 may be inflated as shown in FIG. 6D. In the inflated state, the balloon 368 may affect the targeted anatomy. For instance, in the case of a paranasal sinus ostium, frontal recess, Eustachian tube, or meatus, the inflated the balloon 368 may dilate the anatomical passageway. In the case of a turbinate, the inflated the balloon 368 may crush a posterior nasal nerve in the turbinate and/or otherwise remodel the turbinate. In the case of a deviated septum, the inflated the balloon 368 may straighten the septum. In cases where the balloon 368 is coated with a drug or other material, the inflated the balloon 368 may apply that drug or other material to adjacent tissue. Alternatively, the inflated the balloon 368 may provide various other effects. The inflated the balloon 368 may deform around the guide rail 350, as described above with respect to the shaft assemblies 130, 230. In addition, flexibility in the guide element 370 may allow the guide element 370 to deform when the balloon 368 is inflated and thus bears against the guide element 370, as described above with respect to the shaft assembly 230. The balloon 368 may also deform to some degree around the guide element 370 when the balloon 368 is inflated, as also described above with respect to the shaft assembly 230. After the balloon 368 has been inflated and the desired effects achieved, the operator may deflate the balloon 368 and remove the shaft assembly 330 from the patient.

### II. EXAMPLES OF COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example 1: An apparatus, comprising: (a) a body; and (b) a shaft assembly extending distally relative to the body, the shaft assembly including: (i) an outer sheath defining a central longitudinal axis, (ii) a dilation catheter assembly, the dilation catheter assembly including a dilator, the dilator having a central longitudinal axis, and (iii) a first guide feature, the first guide feature extending distally relative to the outer sheath, the first guide feature having a central longitudinal axis that is laterally offset relative to the central longitudinal axis of the dilator, the dilation catheter assembly being translatable along the first guide feature.

Example 2: The apparatus of Example 1, the body comprising a handle.

Example 3: The apparatus of Example 2, further comprising an actuator, the actuator being movable relative to the handle to drive translation of the dilation catheter assembly along the first guide feature.

Example 4: The apparatus of any of Examples 1 through 3, the outer sheath being rigid.

Example 5: The apparatus of any of Examples 1 through 4, the dilation catheter assembly comprising a proximal shaft portion and a distal shaft portion, the balloon being positioned on the distal shaft portion, at least part of the proximal shaft portion being positioned within the outer sheath.

Example 6: The apparatus of Example 5, the proximal shaft portion having a first outer diameter, the distal shaft portion having a second outer diameter, the first outer diameter being larger than the second outer diameter.

Example 7: The apparatus of any of Examples 5 through 6, the proximal shaft portion defining a first lumen, the first guide feature being slidably disposed in the first lumen.

Example 8: The apparatus of any of Examples 5 through 7, the proximal shaft portion defining a central longitudinal axis, the central longitudinal axis of the first guide feature being laterally offset relative to the central longitudinal axis of the proximal shaft portion.

Example 9: The apparatus of Example 8, the central longitudinal axis of the dilator being laterally offset relative to the central longitudinal axis of the proximal shaft portion.

Example 10: The apparatus of any of Examples 8 through 9, the distal shaft portion having a longitudinal axis that is laterally offset relative to the central longitudinal axis of the proximal shaft portion.

Example 11: The apparatus of Example 8, the central longitudinal axis of the dilator being coaxial with the longitudinal axis of the proximal shaft portion.

Example 12: The apparatus of any of Examples 8 or 11, the distal shaft portion having a longitudinal axis that is coaxial with the central longitudinal axis of the proximal shaft portion.

Example 13: The apparatus of any of Examples 5 through 12, the dilation catheter assembly further comprising a hub, the proximal shaft portion extending proximally relative to the hub, the distal shaft portion extending distally relative to the hub, the hub defining a central longitudinal axis.

Example 14: The apparatus of Example 13, the central longitudinal axis of the hub being coaxial with the central longitudinal axis of the outer sheath.

Example 15: The apparatus of any of Examples 13 through 14, the proximal shaft portion having an outer diameter, the hub having an outer diameter, the outer diameter of the hub being larger than the outer diameter of the proximal shaft portion.

Example 16: The apparatus of any of Examples 13 through 15, the distal shaft portion having an outer diameter, the hub having an outer diameter, the outer diameter of the hub being larger than the outer diameter of the distal shaft portion.

Example 17: The apparatus of any of Examples 13 through 16, the proximal shaft portion and the distal shaft portion together being defined by a single, continuous tubular structure.

Example 18: The apparatus of Example 17, the single, continuous tubular structure passing through the hub.

Example 19: The apparatus of any of Examples 13 through 18, the hub being fixedly secured relative to the proximal shaft portion and the distal shaft portion.

Example 20: The apparatus of any of Examples 13 through 19, the first guide feature being slidably disposed in the hub.

Example 21: The apparatus of any of Examples 13 through 20, the central longitudinal axis of the first guide feature being laterally offset relative to the central longitudinal axis of the hub.

Example 22: The apparatus of any of Examples 1 through 21, the shaft assembly further comprising a second guide feature, the second guide feature having a distal end and an indicator element at or near the distal end.

Example 23: The apparatus of Example 22, the indicator element including a position sensor.

Example 24: The apparatus of Example 23, the position sensor comprising a coil.

Example 25: The apparatus of any of Examples 22 through 24, the indicator element including an illumination source.

Example 26: The apparatus of any of Examples 22 through 25, the second guide feature comprising a guidewire.

Example 27: The apparatus of any of Examples 22 through 26, the second guide feature being disposed in the dilation catheter assembly.

Example 28: The apparatus of Example 27, the second guide feature having a central longitudinal axis that is coaxial with the central longitudinal axis of the dilator.

Example 29: The apparatus of any of Examples 22 through 28, the second guide feature having a central longitudinal axis that is laterally offset relative to the central longitudinal axis of the dilator.

Example 30: The apparatus of any of Examples 22 through 29, the second guide feature being translatable relative to the dilation catheter assembly.

Example 31: The apparatus of any of Examples 1 through 30, the first guide feature comprising a guide rail.

Example 32: The apparatus of Example 31, at least a distal portion of the guide rail having a malleable region.

Example 33: The apparatus of Example 32, the dilation catheter assembly being translatable along the malleable region.

Example 34: The apparatus of Example 33, the malleable region being bendable to an ad hoc bending angle, the guide rail being configured to maintain the ad hoc bending angle as the dilation catheter assembly translates along the malleable region.

Example 35: The apparatus of any of Examples 1 through 34, the dilator comprising a balloon, the balloon being configured to transition between a non-inflated state and an inflated state.

Example 36: The apparatus of Example 35, the balloon being configured to be spaced away from the first guide feature when the balloon is in the non-inflated state.

Example 37: The apparatus of Example 36, the balloon being configured to engage the first guide feature when the balloon is in the inflated state.

Example 38: The apparatus of Example 37, the balloon being configured to deform around the guide feature when the balloon is in the inflated state.

Example 39: The apparatus of any of Examples 1 through 38, the first guide feature being fixedly secured relative to the outer sheath.

Example 40: The apparatus of any of Examples 1 through 39, the first guide feature having a proximal end positioned at or near a distal end of the outer sheath.

Example 41: An apparatus, comprising: (a) a body; and (b) a shaft assembly extending distally relative to the body, the shaft assembly including: (i) an outer sheath defining a central longitudinal axis, (ii) a dilation catheter assembly, the dilation catheter assembly including a balloon, the dilation catheter assembly having a central longitudinal axis, and (iii) a malleable guide rail extending distally relative to the outer sheath, the malleable guide rail having a central longitudinal axis that is laterally offset relative to the central longitudinal axis of the dilator, the dilation catheter assembly being translatable along the malleable guide rail.

Example 42: An apparatus, comprising: (a) a body; and (b) a shaft assembly extending distally relative to the body, the shaft assembly including: (i) an outer sheath defining a central longitudinal axis, (ii) a dilation catheter assembly, the dilation catheter assembly including a balloon, the dilation catheter assembly having a central longitudinal axis, (iii) a first guide feature extending distally relative to the outer sheath, the first guide feature being malleable and having a central longitudinal axis that is laterally offset relative to the central longitudinal axis of the dilator, the dilation catheter assembly being translatable along the first guide feature, and (iv) a second guide feature, the second guide feature having an indicator element, the indicator element including one or both of a position sensor or an illuminating element.

### III. MISCELLANEOUS

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present inventions, further adaptations of the methods and systems described herein may be accomplished by appropriate modifications by one skilled in the art without departing from the scope of the present inventions. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present inventions should be considered in terms of the following claims and is understood not to be limited to the details of structure and operation shown and described in the specification and drawings.

What is claimed is:

1. A medical instrument, comprising a body and a shaft assembly extending distally relative to the body, the shaft assembly comprising:
an outer sheath;
a dilation catheter assembly comprising a dilator positioned along a first longitudinal axis; and
a first guide feature extending distally relative to the outer sheath and defining a second longitudinal axis that is laterally offset relative to the first longitudinal axis, the dilation catheter assembly being translatable along the first guide feature.

2. The medical instrument of claim 1, further comprising an actuator that is movable relative to the body to drive translation of the dilation catheter assembly along the first guide feature.

3. The medical instrument of claim 1, wherein the dilation catheter assembly comprises a distal shaft portion, the dilator being positioned on the distal shaft portion, and a proximal shaft portion, at least part of which is positioned within the outer sheath.

4. The medical instrument of claim 3, wherein the proximal shaft portion comprises a first outer diameter, and the distal shaft portion comprises a second outer diameter that is smaller than the second outer diameter.

5. The medical instrument of claim 3, wherein the proximal shaft portion defines a first lumen, and the first guide feature is slidably disposed in the first lumen.

6. The medical instrument of claim 3, wherein the proximal shaft portion defines a third longitudinal axis that is laterally offset relative to the second longitudinal axis.

7. The medical instrument of claim 6, wherein the first longitudinal axis is offset relative to the third longitudinal axis or coaxial with the third longitudinal axis.

8. The medical instrument of claim 6, wherein the distal shaft portion defines a fourth longitudinal axis that is laterally offset relative to the third longitudinal axis or coaxial with the third longitudinal axis.

9. The medical instrument of claim 3, wherein the dilation catheter assembly further comprises a hub, the proximal shaft portion extends proximally relative to the hub, the distal shaft portion extends distally relative to the hub, and the hub defines a fifth longitudinal axis that is coaxial with the first longitudinal axis.

10. The medical instrument of claim 9, wherein the proximal shaft portion comprises an outer diameter, and the hub comprises an outer diameter that is larger than the outer diameter of the proximal shaft portion.

11. The medical instrument of claim 9, the distal shaft portion having an outer diameter, and the hub comprises an outer diameter that is larger than the outer diameter of the distal shaft portion.

12. The medical instrument of claim 9, wherein the proximal shaft portion and the distal shaft portion define a single, continuous tubular structure that passes through the hub, and the hub is fixedly secured relative to the proximal shaft portion and the distal shaft portion.

13. The medical instrument of claim 9, wherein the first guide feature is slidably disposed in the hub.

14. The medical instrument of claim 9, wherein the second longitudinal axis is laterally offset relative to the fifth longitudinal axis.

15. The medical instrument of claim 1, wherein the shaft assembly further comprises a second guide feature having a distal end and an indicator element at or near the distal end, the indicator element comprising one or more of a position sensor or an illumination source.

16. The medical instrument of claim 15, wherein the second guide feature is disposed in and translatable relative to the dilation catheter assembly.

17. The medical instrument of claim 16, wherein the second guide feature defines a sixth longitudinal axis that is laterally offset relative to the first longitudinal axis or coaxial with the first longitudinal axis.

18. The medical instrument of claim 1, wherein the first guide feature comprises a guide rail, at least a distal portion of which comprises a malleable region; the dilation catheter assembly is translatable along the malleable region, which is bendable to an ad hoc bending angle; and the guide rail is configured to maintain the ad hoc bending angle as the dilation catheter assembly translates along the malleable region.

19. The medical instrument of claim 1, wherein the dilator comprises a balloon configured to:
transition between a non-inflated state and an inflated state;
be spaced away from the first guide feature when the balloon is in the non-inflated state;
engage the first guide feature when the balloon is in the inflated state; and
deform around the guide feature when the balloon is in the inflated state.

20. The medical instrument of claim 1, wherein the first guide feature is fixedly secured relative to the outer sheath.
